SEQUENCE LISTING (2 sequences)

SEQ ID NO: 1 - protein, 390 aa, Flavobacterium okeanokoites
MGFLSMVSKIRTFGWVQNPGKFENLKRVVQVFDRNSKVHNEVKNIKIPTLVKESKIQKELVAIMNQHDLIYTYKELVGTGTSIRSEAPCDAIIQATIADQGNKKGYIDNWSSDGFLRWAHALGFIEYINKSDSFVITDVGLAYSKSADGSAIEKEILIEAISSYPPAIRILTLLEDGQHLTKFDLGKNLGFSGESGFTSLPEGILLDTLANAMPKDKGEIRNNWEGSSDKYARMIGGWLDKLGLVKQGKKEFIIPTLGKPDNKEFISHAFKITGEGLKVLRRAKGSTKFTRVPKRVYWEMLATNLTDKEYVRTRRALILEILIKAGSLKIEQIQDNLKKLGFDEVIETIENDIKGLINTGIFIEIKGRFYQLKDHILQFVIPNRGVTKGT

SEQ ID NO: 2 - protein, 196 aa, Flavobacterium okeanokoites
KKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFGT

(19) United States
(12) Patent Application Publication
(10) Pub. No.: US 2008/0050743 A1
(43) Pub. Date: Feb. 28, 2008

(54) Binary signal detection assays

(75) Inventors: Joseph A. Sorge, Wilson, WY (US); Carsten-Peter Carstens, San Diego, CA (US); Craig Robert Monell, La Jolla, CA (US); Alexander Belyaev, San Diego, CA (US)

Correspondence Address: AGILENT TECHOLOGIES INC, P.O BOX 7599, BLDG E , LEGAL, LOVELAND, CO, 80537-0599, US

(73) Assignee: Stratagene California, La Jolla, CA (US)

(21) Appl. No.: 11/821,286

(22) Filed: Jun. 22, 2007

Related U.S. Application Data
(63) Continuation of application No. 11/546,695, filed on Oct. 11, 2006, now abandoned.
(60) Provisional application No. 60/725,990, filed on Oct. 11, 2005.
(60) Provisional application No. 60/754,001, filed on Dec. 23, 2005.

(51) Int. Cl. C12Q 1/68

(52) U.S. Cl. 435/6

(57) ABSTRACT

The present invention provides methods, kits and compositions for the detection of an analyte. The invention is particularly suited for the detection and quantification of analytes in solution. In the methods of the invention a complex is formed between two or more analyte specific probes (ASP) and an analyte. The reactive moieties of the probes interact upon the binding of the analyte specific probes to the analyte. The reactive moieties generate a nucleic acid cleavage product which is detected and indicative of the presence of the analyte.

### RELATED APPLICATIONS

This application is a continuation application of U.S. application Ser. No. 11/546,695, filed Oct. 11, 2006, which claims the benefit of U.S. Provisional Application No. 60/725,990, filed on Oct. 11, 2005 and U.S. Provisional Application No. 60/754,001, filed on Dec. 23, 2005. The entire teachings of the above applications are incorporated herein by reference.

### BACKGROUND

The development of immunoassays and advances in nucleic acid detection have advanced the art of the detection of biological samples. Several so called “proximity-probe” assays are known in the art. Proximity-probes produce a detectable signal when the probes bind an analyte within close proximity to each other. Such assays are described in U.S. Publication No. 2002/0064779, U.S. Pat. No. 6,511,809, U.S. Publication No. 2005/0003361, PCT publication WO 2005/019470.

### SUMMARY OF THE INVENTION

The present invention provides methods, kits and compositions for the detection of an analyte. The invention is particularly suited for the detection and quantification of analytes in solution. In the methods of the invention a complex is formed between two or more analyte specific probes (ASP) and an analyte. The reactive moieties of the probes interact upon the binding of the analyte specific probes to the analyte. The reactive moieties generate a nucleic acid cleavage product which is detected and indicative of the presence of the analyte.

In a first aspect of the invention, the invention provides a method for detecting an analyte with a first and second analyte specific probe and a cleavage agent. The first analyte specific probe comprises a first binding moiety and an oligonucleotide. The second analyte specific probe comprises a second binding moiety and a second oligonucleotide. In one embodiment, the first and second oligonucleotides are completely complementary. In another embodiment, the first and second oligonucleotides are partially complementary. The first and the second binding moieties of the probe bind to the analyte. As a result of the two probes binding to the analyte, within close proximity to each other, a cleavage site is formed so as to permit cleavage at the cleavage site with the cleavage agent thereby releasing a cleavage product. The cleavage product is detected and is indicative of the presence of the analyte.

In a related aspect of the invention, the invention provides kits and compositions related to the previous aspect. The kits and compositions include a first analyte specific probe comprising a first binding moiety and an oligonucleotide, a second analyte specific probe comprising a second binding moiety and a second oligonucleotide and packaging material therefore. The first and second binding moieties bind to the analyte to form a cleavage site.

In another aspect of the invention, the invention provides a method for detecting an analyte by providing an analyte and a first and second analyte specific probe. The first analyte specific probe comprises a first binding moiety and an oligonucleotide having a cleavage site. In one embodiment, the oligonucleotide is at least partially complementary to itself. The second analyte specific probe comprises a second binding moiety and a cleavage agent. The first and the second binding moieties of the probe bind to the analyte. As a result of the two or more probes binding to the analyte, within close proximity to each other, the oligonucleotide and the cleavage agent interact to form a complex having the oligonucleotide and the cleavage agent. The cleavage agent cleaves the oligonucleotide at the cleavage site thereby releasing a cleavage product. The cleavage product is detected and is indicative of the presence of the analyte.

In a related aspect of the invention, the invention provides kits and compositions for detecting an analyte. The kits and compositions include a first analyte specific probe having a first binding moiety, an oligonucleotide which has a cleavage site and packaging material therefore. The kits and compositions also include a second analyte specific probe having a second binding moiety and a cleavage agent. The first and second binding moieties bind to the analyte to form a complex of the oligonucleotide and the cleavage agent.

In yet another aspect of the invention, the invention provides a method for detecting an analyte by providing an analyte and a first and second analyte specific probe. The first analyte specific probe comprises a first binding moiety and an oligonucleotide having a cleavage site, cleavage activity and an activator binding site. In one embodiment, the oligonucleotide is a DNAzyme. In another embodiment, the oligonucleotide is a ribozyme. The second analyte specific probe includes a second binding moiety and an activator. The first and the second binding moieties of the probe bind to the analyte. As a result of the two probes binding to the analyte, within close proximity to each other, the oligonucleotide and the activator interact allowing the activator to bind the activator binding site and activating the cleavage activity in the oligonucleotide, e.g., DNAzyme. The cleavage activity cleaves the oligonucleotide at the cleavage site thereby releasing a cleavage product. The cleavage product is detected and is indicative of the presence of the analyte.

In a related aspect of the invention, the invention provides kits and compositions for detecting an analyte by the method of the previous aspect. The kits and compositions include a first analyte specific probe having a first binding moiety, an oligonucleotide having a cleavage site, cleavage activity and an activator binding site and packaging material therefore. The kits and compositions also include a second analyte specific probe having a second binding moiety and an activator. The oligonucleotide and activator interact when the first binding moiety and the second binding moiety bind the analyte.

In yet another aspect of the invention, the invention provides a method for detecting an analyte by providing an analyte, a first and a second analyte specific probe and a cleavage agent. The first analyte specific probe comprises a first binding moiety and an oligonucleotide. In one embodiment, the 3′ end of the oligonucleotide is at least partially annealed to the oligonucleotide. The second analyte specific probe comprises a second binding moiety and a polymerase. The first and the second binding moieties of the probe bind to the analyte. As a result of the two or more probes binding to the analyte, within close proximity to each other, the oligonucleotide and the polymerase interact so that the polymerase synthesizes a nucleic acid strand and forms a cleavage site. The cleavage agent cleaves the oligonucleotide at the cleavage site thereby releasing a cleavage product. The cleavage product is detected and is indicative of the presence of the analyte.

In a related aspect of the invention, the invention provides kits and compositions for detecting an analyte by the method of the previous aspect. The kits and compositions include a first analyte specific probe having a first binding moiety and an oligonucleotide. The kits and compositions also include a second analyte specific probe comprising a second binding moiety and a polymerase. The oligonucleotide and the polymerase interact when the first binding moiety and the second binding moiety bind the analyte.

In another aspect of the invention, the invention provides a method of detecting an analyte by incubating a mixture comprising a first analyte specific probe and a second analyte specific probe. The first analyte specific probe includes a first binding moiety and a DNA binding domain and the second analyte specific probe includes a second binding moiety and a cleavage agent. The first and second binding moieties bind to the analyte allowing the DNA binding domain and cleavage agent to interact so as to permit cleavage of a target nucleic acid. The cleavage agent cleaves the target nucleic acid thereby releasing a cleavage product. The cleavage product is detected and is indicative of the presence of the analyte.

In a related aspect of the invention, the invention provides kits and compositions for detecting an analyte by the method of the previous aspect. The kits and compositions include a first analyte specific probe having a first binding moiety and a DNA binding domain and a second analyte specific probe having a second binding moiety and a cleavage agent.

In another aspect of the invention, the invention provides for a method for detecting an analyte by incubating a mixture comprising a first analyte specific probe and a second analyte specific probe, and a target nucleic acid. The first analyte specific probe includes a first binding moiety and a first portion of a cleavage agent and the second analyte specific probe includes a second binding moiety and a second portion of a cleavage agent. The first and second portions of the cleavage agents have no or reduced cleavage activity when compared to the cleavage activity when the first and second portions of the cleavage agents interact, e.g., when the first and second binding moieties bind to the same analyte. The first and second biding moieties bind to the analyte to allow the first portion and second portion of the cleavage agent to interact to form a cleavage agent with increased cleavage activity. The cleavage activity cleaves a target nucleic acid at a cleavage site, thereby releasing a cleavage product. The cleavage product is detected and is indicative of the presence of the analyte.

In a related aspect of the invention, the invention provides kits and compositions for detecting an analyte by the method of the previous aspect. The kits and compositions include a first analyte specific probe having a first binding moiety and a first portion of a cleavage agent and a second analyte specific probe having a second binding moiety and a second portion of a cleavage agent.

### BRIEF DESCRIPTION OF THE DRAWINGS

FIG. 1 is a diagram illustrating one embodiment of the two oligonucleotide ASP detection method.

FIG. 2 is a diagram illustrating one embodiment of the single oligonucleotide-cleavage agent ASP detection method.

FIG. 3 is a diagram illustrating one embodiment of the DNAzyme/Ribozyme-Activator ASP detection method.

FIG. 4 is a diagram illustrating one embodiment of the single oligonucleotide-polymerase ASP detection method.

FIG. 5 is a diagram illustrating one embodiment of the DNA binding domain-cleavage agent ASP detection method.

FIG. 6A is the amino acid sequence of a first portion of a cleavage agent which includes the Fok I DNA binding domain.

FIG. 6B is the amino acid sequence of a second portion of a cleavage agent which includes the Fok I cleavage domain.

### DETAILED DESCRIPTION

Definitions

As used herein the term “analyte” refers to a substance to be detected or assayed by the method of the present invention. Typical analytes may include, but are not limited to proteins, peptides, cell surface receptor, receptor ligand, nucleic acids, molecules, cells, microorganisms and fragments thereof, or any substance for which a binding moiety, e.g., antibodies, can be developed.

As used herein the term “binding moiety” refers to a molecule which stably binds an analyte. Binding moieties include, but are not limited, to a monoclonal antibody, polyclonal antibody, aptamer, cell surface receptor, receptor ligand, biotin, streptavidin, avidin, protein A and protein G and binding fragments thereof, e.g., Fab. The binding moiety is directly or indirectly coupled to a reactive molecule.

As used herein, the term “antibody” refers to an immunoglobulin protein which is capable of binding an antigen, e.g., analyte. Antibody as used herein is meant to include antibody fragments (e.g., F(Ab′)₂, FAb′, FAb, Fv, scFv) capable of binding the analyte of interest.

The terms “specifically binds” or “specific”, as used herein in reference to a binding moiety to an analyte, means the recognition, contact, and formation of a stable complex between the ASP's binding moiety and an analyte, together with substantially less recognition, contact, or complex formation of the ASP with other molecules

As used herein the terms “analyte specific probe” or “ASP”, refers to a molecule having a binding moiety and a reactive moiety (e.g., a nucleic acid, enzyme, activating agent, etc.). The binding moiety is operatively coupled to the reactive moiety. The analyte specific probes require that two or more probes bind in close proximity to one another in order for the reactive moieties to effectively interact. The analyte specific probes are in close proximity to one another when the two probes bind to their respective binding sites on the analyte.

As used herein, the term “oligonucleotide” refers polydeoxyribonucleotides (containing 2-deoxy-D-ribose), to polyribonucleotides (containing D-ribose) and to any polynucleotide which is an N-glycoside of a purine or pyrimidine base, or modified purine or pyrimidine base. An oligonucleotide may hybridize to other oligonucleotide or may self-hybridize, e.g., hairpin structure. An oligonucleotide includes, without limitation, single- and double-stranded oligonucleotides. The term “oligonucleotide” as it is employed herein embraces chemically, enzymatically or metabolically modified forms of oligonucleotides.

As used herein a “cleavage agent” refers to a polypeptide that is specific for, that is, cleaves a cleavage site according to the invention and is not specific for, that is, does not substantially cleave an oligonucleotide that does not have a cleavage site. Cleavage agent as used herein is meant to include fragments of cleavage enzymes capable of cleaving a cleavage site. Cleavage agents include restriction enzymes, nucleases, nickases, ribozymes, DNAzymes and fragments thereof.

As used herein, a “cleavage site” refers to a polynucleotide structure or sequence that is capable of being cleaved by a cleavage agent. Cleavage sites include, but are not limited to, restriction enzyme sites, ribozyme sites, nickase sites, DNAzyme sites and nuclease cleavage sites.

As used herein, the term “nuclease cleavage site” refers to a structure within an oligonucleotide that is susceptible to cleavage by a nuclease. Such sites are known in the art and are described herein. In one embodiment, the cleavage site comprises at least a duplex nucleic acid having a single stranded region comprising a flap, a loop, a single-stranded bubble, a D-loop, a nick or a gap. A cleavage site according to this embodiment of the invention includes a polynucleotide structure comprising a flap strand of a branched DNA wherein a 5′ single-stranded polynucleotide flap extends from a position near its junction to the double stranded portion of the structure. Such cleavage structures are described in U.S. Pat. Nos. 6,548,250, 6,348,314 and 6,528,254, which are herein incorporated by reference in their entirety.

The term “nuclease” includes an enzyme that possesses 5′ endonucleolytic activity for example a DNA polymerase, e.g. DNA polymerase I from E. coli, and DNA polymerase from Thermus aquaticus (Taq), Thermus thermophilus (Tth), Pyrococcusfuriosus (Pfu) and Thermus flavus (Tfl). The term “nuclease” also embodies FEN nucleases.

As used herein, the term “restriction enzyme” refers to an enzyme which cuts double-stranded DNA at or near a specific nucleotide sequence. The specificities of numerous restriction enzymes are well known in the art. Various restriction enzymes are commercially available and their reaction conditions, cofactors, and other requirements as established by the enzyme suppliers are well known.

As used herein, the term “interact” as it refers to the reactive moieties of the ASP, refers to bringing at least a pair of reactive moieties of an analyte specific probe within close proximity to on one another so the reactive moieties form a physical interaction. For example, when a pair of analyte specific probes having a first and second oligonucleotide bind to an analyte the first and second oligonucleotides are brought into close proximity so as to interact, e.g., anneal to each other. In another embodiment, a pair of analyte specific probes binds to an analyte in which a first ASP has an oligonucleotide having a cleavage site and a second ASP has a cleaving agent. In this embodiment, the oligonucleotide and the cleavage agent are brought into close proximity to one another so as to interact. Thus, the cleaving agent binds to and cleaves the first oligonucleotide at the cleavage site. In yet another embodiment, a pair of analyte specific probes bind to an analyte, an oligonucleotide on a first ASP and an activator on a second ASP are brought into close proximity to one another. The activator binds to or interacts with the oligonucleotide and activates a cleavage activity of the oligonucleotide. In yet another embodiment, a pair of analyte specific probes bind to an analyte, in which the first ASP has a first oligonucleotide and the second ASP is coupled to a polymerase. When bound to the same analyte the first oligonucleotide and the polymerase interact or bind resulting in the polymerase extending a 3′ end of the oligonucleotide.

As used herein, the term “cleavage product” is an oligonucleotide fragment that has been cleaved and released into solution in a cleavage reaction by a cleaving agent. In some embodiments, the cleavage product is an oligonucleotide cleaved by a nuclease. In another embodiment, the cleavage product is an oligonucleotide cleaved by a restriction enzyme. In most detection methods, the cleavage product is complementary to and hybridize with an additional nucleic acid of a downstream detection assay. In some embodiments, the cleavage product is a primer or probe in a downstream detection assay, e.g., sequential amplification reaction.

As used herein, “cleavage reaction” refers to enzymatically separating an oligonucleotide (i.e. not physically linked to other fragments or nucleic acids by phosphodiester bonds) into fragments or nucleotides and fragments that are released from the oligonucleotide. A cleavage reaction is performed by an exonuclease activity, endonuclease activity or restriction enzyme activity. Cleavage reactions utilizing an endonuclease activity are described in U.S. Pat. Nos. 6,548,250, 5,210,015, 6,348,314 and 6,528,254, which are herein incorporated by reference in their entirety. Cleavage reaction assays encompassed by the present methods also include assays utilizing exonuclease activity such as those described in U.S. Pat. No. 5,723,591, which is herein incorporated by reference in its entirety. Such cleavage reactions may be practiced by the cleavage agent in the methods of the invention or in subsequent detection steps utilizing the released cleavage product.

As used herein, the term “sequential amplification reaction” and “sequential cleavage reaction” refer to a reaction in which the cleavage product is utilized as a primer or probe (or invader oligonucleotide) in a subsequent detection reaction generating one or more additional cleavage products which produce a detectable signal. Sequential amplification reactions include INVADER technology assays (Third Wave Technologies, Madison, Wis.) and those cleavage assays described U.S. Pat. No. 6,893,819 and 6,348,314, both of which are herein incorporated by reference in their entireties, as well as other such methods known in the art and described herein.

As used herein, the term “activator” refers to a molecule which activates the cleavage activity of a DNAzyme or Ribozyme. Such molecules may include metal ions, oligonucleotides and small molecules. The activator binds the activator binding site on the DNAzyme or ribozyme, thus activating the cleavage activity.

As used herein, the term “DNA binding domain” refers to a stretch of amino acids which is capable of directing polypeptide binding to a particular DNA sequence. The binding may be specific for a particular nucleic acid sequence. Suitable DNA binding domains are well known in the art. In one embodiment, the DNA binding domain is a Ubx homeodomain. In another embodiment, the DNA binding domain is a GAL4 DNA binding domain. In yet another embodiment, the DNA binding domain is AlwI DNA binding domain. In still another embodiment, the DNA binding domain is a Zif-QQR-F_{N} DNA binding domain. In another embodiment, the DNA binding domain is a ZIF-ΔQNK-F_{N} DNA binding domain.

As used herein, the term “pair of interacting domains” refers to a set of polypeptides or nucleic acids which specifically bind to each other. Pairs of interacting nucleic acid domains include complementary sequences of nucleic acids, e.g., 4-30 bases, 5-20 bases, or 6-15 bases. Pairs of interacting polypeptides can be any known in the art and can include dimerization domains selected from the group consisting of jun/fos, jun/jun, SH2 (src homology 2), SH3 (src Homology 3), phosphotyrosine binding (PTB), WW, PDZ, 14.3.3, WD40, EH, Lim, and the like, and can further comprise mutants of these domains in which the affinity is altered. The polypeptide pairs can be identified by methods known in the art, including yeast two hybrid screens. Yeast two hybrid screens are described in U.S. Pat. Nos. 5,283,173 and 6,562,576, both of which are herein incorporated by reference in their entireties. Affinities between a pair of interacting domains can be determined using methods known in the art, including as described in Katahira et al. (2002) J Biol Chem. 277, 9242-9246, incorporated herein by reference.

As used herein, the term “free domain” refers to a polypeptide or nucleic acid which interacts with one or the other of the pair of interacting domains, but which is not part of an analyte specific probe, and is otherwise not capable of generating a signal that is detected in the methods used. In one embodiment, the “free domain” is a nucleic acid or polypeptide which is identical to one or the other of the interacting domains used. For example, if a jun/fos pair of interacting domains is used in the first and second analyte probe, a polypeptide consisting of either the jun or fos dimerization domain can be used as a “free domain.”

As used herein, the term “complementary” refers to the concept of sequence complementarity between regions of two polynucleotide/oligonucleotide strands. It is known that an adenine base of a first polynucleotide region is capable of forming specific hydrogen bonds (“base pairing”) with a base of a second polynucleotide region which is antiparallel to the first region if the base is thymine or uracil. Similarly, it is known that a cytosine base of a first polynucleotide strand is capable of base pairing with a base of a second polynucleotide strand which is antiparallel to the first strand if the base is guanine. A first region of a polynucleotide is complementary to a second region a different polynucleotide if, when the two regions are arranged in an antiparallel fashion, at least one nucleotide of the first region is capable of base pairing with a base of the second region. Therefore, it is not required for two complementary polynucleotides to base pair at every nucleotide position. “Complementary” can refer to a first polynucleotide that is 100% or “fully” complementary to a second polynucleotide and thus forms a base pair at every nucleotide position. “Complementary” also can refer to a first polynucleotide that is not 100% complementary (e.g., 90%, 80%, 70% complementary or less) contains mismatched nucleotides at one or more nucleotide positions.

As used herein, the terms “hybridization” or “annealing” is used to describe the pairing of complementary (including partially complementary) polynucleotide/oligonucleotide strands, e.g., a first and second oligonucleotide of an ASP. Hybridization and the strength of hybridization (i.e., the strength of the association between polynucleotide strands) is impacted by many factors well known in the art including the degree of complementarity between the polynucleotides, stringency of the conditions involved, the melting temperature (Tₘ) of the formed hybrid, the presence of other components (e.g., the presence or absence of polyethylene glycol), the molarity of the hybridizing strands, and the G:C content of the polynucleotide strands.

As used herein, when one polynucleotide is said to “hybridize” to another polynucleotide, it means that there is some complementarity between the two polynucleotides or that the two polynucleotides form a hybrid under high stringency conditions. When one polynucleotide is said to not hybridize to another polynucleotide, it means that there is no sequence complementarity between the two polynucleotides or that no hybrid forms between the two polynucleotides at a high stringency condition.

As used herein, “nucleic acid polymerase” or “polymerase” refers to an enzyme that catalyzes the polymerization of nucleotides. Generally, the enzyme will initiate synthesis at the 3′-end of the primer annealed to a nucleic acid template sequence, and will proceed toward the 5′ end of the template strand. “DNA polymerase” catalyzes the polymerization of deoxyribonucleotides. Known DNA polymerases include, for example, Pyrococcus furiosus (Pfu) DNA polymerase (Lundberg et al., 1991, Gene, 108:1), E. coli DNA polymerase I (Lecomte and Doubleday, 1983, Nucleic Acids Res. 11:7505), T7 DNA polymerase (Nordstrom et al., 1981, J. Biol. Chem. 256:3112), Thermus thermophilus (Tth) DNA polymerase (Myers and Gelfand 1991, Biochemistry 30:7661), Bacillus stearothermophilus DNA polymerase (Stenesh and McGowan, 1977, Biochim Biophys Acta 475:32), Thermococcus litoralis (Tli) DNA polymerase (also referred to as Vent DNA polymerase, Cariello et al., 1991, Nucleic Acids Res, 19: 4193), 9° Nm DNA polymerase (discontinued product from New England Biolabs), Thermotoga maritima (Tma) DNA polymerase (Diaz and Sabino, 1998 Braz J. Med. Res, 31:1239), Thermus aquaticus (Taq) DNA polymerase (Chien et al., 1976, J. Bacteoriol, 127: 1550), Pyrococcus kodakaraensis KOD DNA polymerase (Takagi et al., 1997, Appl. Environ. Microbiol. 63:4504), JDF-3 DNA polymerase (Patent application WO 0132887), and Pyrococcus GB-D (PGB-D) DNA polymerase (Juncosa-Ginesta et al., 1994, Biotechniques, 16:820). The polymerase activity of any of the above enzyme can be determined by means well known in the art. One unit of DNA polymerase activity, according to the subject invention, is defined as the amount of enzyme which catalyzes the incorporation of 10 nmoles of total dNTPs into polymeric form in 30 minutes at optimal temperature (e.g., 72° C. for Pfu DNA polymerase).

Description

The present invention provides methods, kits and compositions for the detection of an analyte. In the methods of the invention, a complex is formed between two or more analyte specific probes and an analyte. The analyte specific probes comprise a binding moiety and a reactive moiety. The reactive moieties of the probes interact upon the binding of the analyte specific probes to the analyte allowing the reactive moieties to generate a nucleic acid cleavage product. The cleavage product is detected in a secondary reaction.

Two Oligonucleotide ASP Detection Method

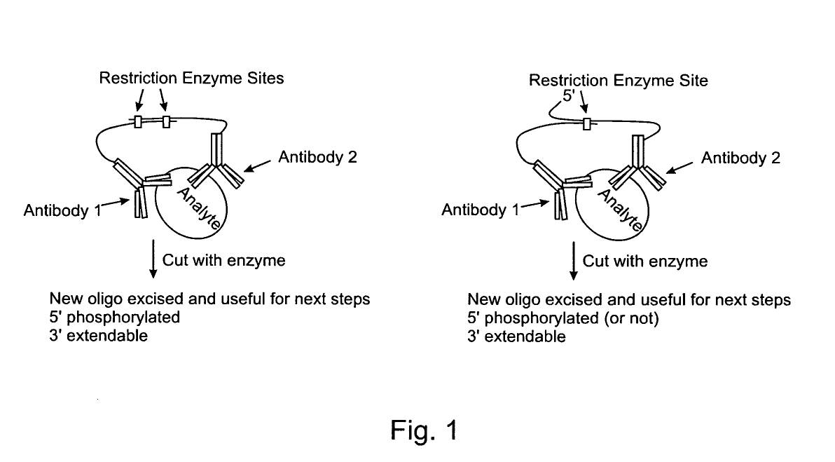

One aspect of the invention is illustrated in FIG. 1. In this aspect a first and second analyte specific probe, an analyte and a cleavage agent are added to a reaction mixture. In some embodiments, a third oligonucleotide is added to the reaction mixture, wherein the third oligonucleotide hybridizes to the first and/or second oligonucleotide. In one embodiment the analyte is a polypeptide. In other embodiments, the analyte is an oligonucleotide, cell surface receptor or receptor ligand.

The first analyte specific probe comprises a first binding moiety and an oligonucleotide, while the second analyte specific probe comprises a second binding moiety and a second oligonucleotide. In the embodiment depicted in FIG. 1, the binding moiety is an antibody. However, binding moieties may also include, but are not restricted to, a lectin, cell surface receptor, receptor ligand, peptide, carbohydrate, aptamer, biotin, streptavidin, avidin, protein A and protein G or binding fragments thereof.

The first and the second binding moieties of the probe bind to the analyte at analyte binding sites. As a result of the two or more probes binding to the same analyte, or to different analytes but within close proximity to each other, the first and second oligonucleotides interact form at least one cleavage site. The oligonucleotides must be of a sufficient length and sufficient complementarity to hybridize.

In one embodiment, the first and second oligonucleotides are completely complementary to one another. In another embodiment, the first and second oligonucleotides are partially complementary to one another. In yet another embodiment, the first and second oligonucleotides interact via hybridization to form a cleavage site. In one embodiment, either the first or the second oligonucleotide has a 5′ flap. In yet a further embodiment, the first oligonucleotide's 5′ flap is complementary to the second oligonucleotide. In another embodiment, the first oligonucleotide's 5′ flap is non-complementary to the second oligonucleotide. In yet another embodiment, the second oligonucleotide's 5′ flap is complementary to the first oligonucleotide. In yet another embodiment, the second oligonucleotide's 5′ flap is non-complementary to the first oligonucleotide.

The cleavage site is susceptible to cleavage by a cleavage agent. In one embodiment, the cleavage site is a restriction enzyme cleavage site. In another embodiment, the cleavage site is a nuclease or FEN cleavage site. In yet other embodiment, the cleavage site is a ribozyme or DNAzyme cleavage site.

The cleavage agent can be a restriction enzyme. In another embodiment, the cleavage agent is a nuclease or FEN. In yet other embodiment, the cleavage agent is a ribozyme or DNAzyme.

The cleavage agent cleaves the oligonucleotide at the cleavage site thereby releasing a cleavage product. The cleavage product is a short nucleic acid fragment from one or both oligonucleotides. In one embodiment, the cleavage product is phosphorylated at its 5′ end and has a 3′ end that is extendable by a polymerase.

The cleavage product is detected and is indicative of the presence of an analyte. In one embodiment, the release of the cleavage product produces a directly detectable signal, (e.g., a change in a detectable signal, for example upon the cleavage and separation of a FRET pair).

In another embodiment, the released cleavage product is detected by a secondary detection assay. Secondary detection assays are those in which the cleavage product acts as a probe or primer in an assay that produces a detectable signal. Such assays include, but are not limited to, linear and exponential signal detection assays such as those taught in U.S. Pat. Nos. 6,893,819 and 6,348,314, INVADER technology assays (Third Wave Technologies, Inc, Madison, Wis.), Padlock probe assays such as those disclosed in U.S. Publication No. 2005/0026180, and other methods known in the art and described herein.

In some secondary detection assays the released cleavage product is extended by a polymerase. In other secondary detection assays the released product acts as a probe and may be cleaved by a second cleavage agent.

In another aspect of the invention, the invention provides kits and compositions related to the two oligonucleotide ASP detection method. The kits and compositions include a first analyte specific probe comprising a first binding moiety and an oligonucleotide, a second analyte specific probe comprising a second binding moiety and a second oligonucleotide. The first and second binding moieties bind to the analyte to form a cleavage site. The compositions and kits may further include a cleavage agent.

Single Oligonucleotide-Cleavage Agent ASP Detection Method

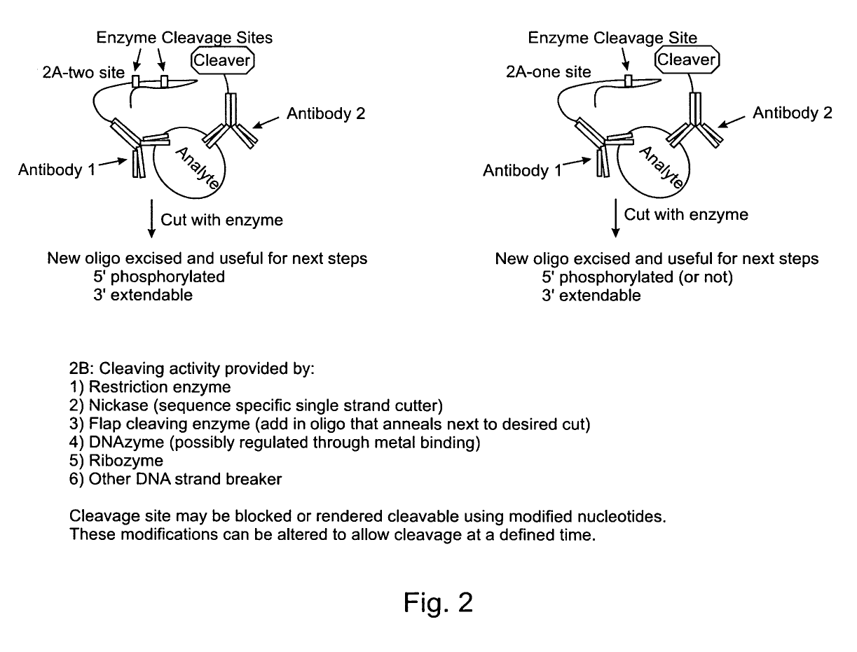

Another aspect of the invention is depicted in FIG. 2. In this aspect a first and second analyte specific probe and an analyte are added to a reaction mixture. In some embodiments, a second oligonucleotide, which hybridizes to the oligonucleotide, is added to the reaction mixture. In one embodiment, the analyte is a polypeptide. In other embodiments, the analyte is an oligonucleotide, cell surface receptor or receptor ligand.

The first analyte specific probe has a first binding moiety and an oligonucleotide having a cleavage site. In one embodiment of the oligonucleotide, the oligonucleotide is at least partially complementary to itself. In another embodiment, the oligonucleotide has a cleavable 5′ flap. The cleavable 5′ flap may be complementary or non-complementary to the oligonucleotide. The cleavage site may be formed by a region of self complementarity, e.g., hairpin structure. The oligonucleotide is operatively coupled to the first binding moiety. The oligonucleotide may be directly or indirectly coupled to the binding moiety. The second analyte specific probe has a second binding moiety and a cleavage agent. The cleavage agent is operatively coupled to the second binding moiety. The cleavage agent may be directly or indirectly coupled to the binding moiety. The binding moieties may be, but are not restricted to, an antibody, a lectin, cell surface receptor, receptor ligand, peptide, carbohydrate, aptamer, biotin, streptavidin, avidin, protein A and protein G or binding fragments thereof.

In another embodiment, a third analyte specific probe is provided having a third binding moiety and a second cleaving agent. In yet another embodiment, a third analyte specific probe has a third binding moiety and a DNA kinase. In a further embodiment, the DNA kinase phosphorylates the 5′ end of the cleavage product.

The first and the second binding moieties of the probe bind to the analyte. As a result of the two or more probes binding to the analyte, within close proximity to each other, the oligonucleotide and the cleavage agent interact to form a complex comprising the oligonucleotide and the cleaving agent. In one embodiment, the cleavage agent is a restriction enzyme. In another embodiment, the cleavage agent is a nuclease or FEN. In yet other embodiments, the cleavage agent is a ribozyme, nickase or DNAzyme cleavage agent.

The cleavage agent cleaves the oligonucleotide at the cleavage site thereby releasing a cleavage product. The cleavage product is a short nucleic acid fragment of the oligonucleotide. In one embodiment, the cleavage product is phosphorylated at its 5′ end and has a 3′ end that is extendable by a polymerase.

The cleavage product is detected and is indicative of the presence of an analyte. In one embodiment, the release of the cleavage product produces a directly detectable signal, (e.g., a change in a detectable signal, for example upon the cleavage and separation of a FRET pair).

In another embodiment, the released cleavage product is detected by a secondary detection assay. Secondary detection assays are those in which the cleavage product acts as a probe or primer in an assay that produces a detectable signal. Such assays include, but are not limited to, linear and exponential signal detection assays such as those taught in U.S. Pat. No. 6,893,819, INVADER technology assays (Third Wave Technologies, Inc, Madison, Wis.), Padlock probe assays such as those disclosed in U.S. Publication No. 2005/0026180, and other methods known in the art and described herein.

In some secondary detection assays the released cleavage product is extended by a polymerase. In other secondary detection assays the released product acts as a probe and may be cleaved by a second cleavage agent.

In a related aspect of the invention, the invention provides kits and compositions for practicing the single oligonucleotide-cleavage agent ASP detection method. The kits and compositions include a first analyte specific probe having a first binding moiety and an oligonucleotide which has a cleavage site. The kits and compositions also include a second analyte specific probe having a second binding moiety and a cleavage agent. The oligonucleotide and the cleavage agent interact to form a complex when the first binding moiety and the second binding moiety bind the analyte.

DNAzyme/Ribozyme-Activator ASP Detection Method

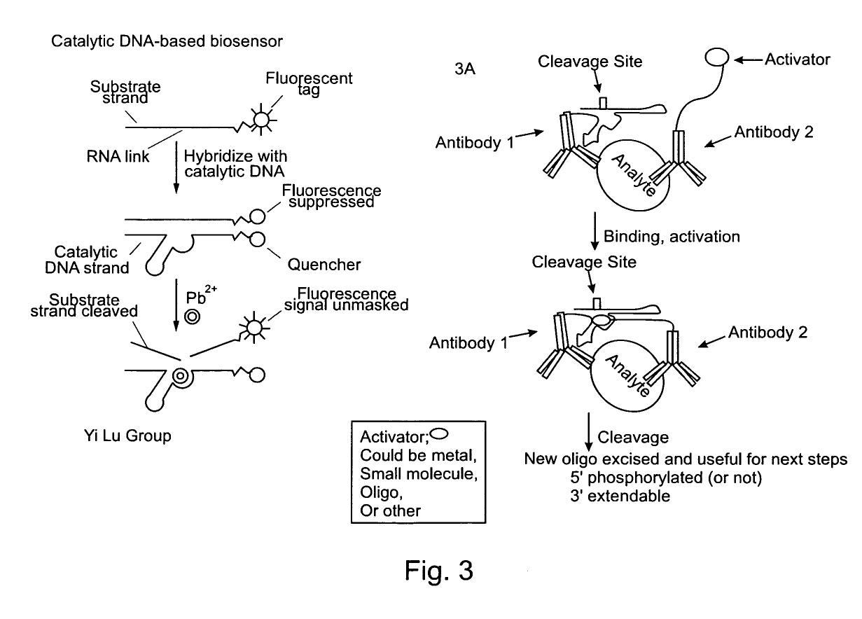

Another aspect of the invention is depicted in FIG. 3. The invention provides a method for detecting an analyte by providing an analyte and a first and second analyte specific probes to a reaction mixture. In some embodiments, a second oligonucleotide is added. The analyte can be a polypeptide, an oligonucleotide, cell surface receptor or receptor ligand.

The first analyte specific probe comprises a first binding moiety and an oligonucleotide having a cleavage site, cleavage activity and activator binding site. In one embodiment, the oligonucleotide is a DNAzyme or ribozyme. In another embodiment, the oligonucleotide does not have a cleavage site but a second oligonucleotide that is added to the reaction mixture has a cleavage site. In one embodiment, the cleavage site recognized and cleaved by a DNAzyme. In another embodiment, the cleavage site is recognized and cleaved by a ribozyme.

The second analyte specific probe includes a second binding moiety and an activator. Activators include, but are not limited to, metal ions, oligonucleotides and small molecules. The activator is a necessary co-factor for the cleavage agent's cleavage activity.

The binding moiety may be, but is not restricted to, an antibody, a lectin, cell surface receptor, receptor ligand, peptide, carbohydrate, aptamer, biotin, streptavidin, avidin, protein A and protein G or binding fragments thereof.

The first and the second binding moieties of the probe bind to the analyte. As a result of the two or more probes binding to the analyte within close proximity to each other, the oligonucleotide and the activator interact allowing the activator to bind the activator binding site and activating the cleavage activity of the oligonucleotide.

The cleavage activity cleaves the oligonucleotide at the cleavage site thereby releasing a cleavage product. The cleavage product is a short nucleic acid fragment of the oligonucleotide. In one embodiment, the cleavage product is phosphorylated at its 5′ end and has a 3′ end that is extendable by a polymerase.

The cleavage product is detected and is indicative of the presence of an analyte. In one embodiment, the release of the cleavage product produces a directly detectable signal, (e.g., a change in a detectable signal, for example upon the cleavage and separation of a FRET pair).

In another embodiment, the released cleavage product is detected by a secondary detection assay. Secondary detection assays are those in which the cleavage product acts as a probe or primer in an assay that produces a detectable signal. Such assays include, but are not limited to, linear and exponential signal detection assays such as those taught in U.S. Pat. Nos. 6,893,819 and 6,348,314, INVADER technology assays (Third Wave Technologies, Inc, Madison, Wis.), Padlock probe assays such as those disclosed in U.S. Publication No. 2005/0026180, and other methods known in the art and described herein.

In some secondary detection assays the released cleavage product is extended by a polymerase. In other secondary detection assays the released product acts as a probe and may be cleaved by a second cleavage agent.

In a related aspect of the invention, the invention provides kits and compositions for performing the DNAzyme/ribozyme-activator ASP detection method. The kits and compositions include a first analyte specific probe having a first binding moiety and an oligonucleotide having a cleavage site, activator binding site and cleavage activity. In some embodiments, the oligonucleotide does not have a cleavage site but a second oligonucleotide has a cleavage site. The kits and compositions also include a second analyte specific probe having a second binding moiety and an activator. The oligonucleotide and activator interact allowing the activator to bind the activator binding site and activate cleavage activity when the first binding moiety and the second binding moiety bind the analyte. The kits and compositions may further include one or more additional oligonucleotides.

Single Oligonucleotide-Polymerase ASP Detection Method

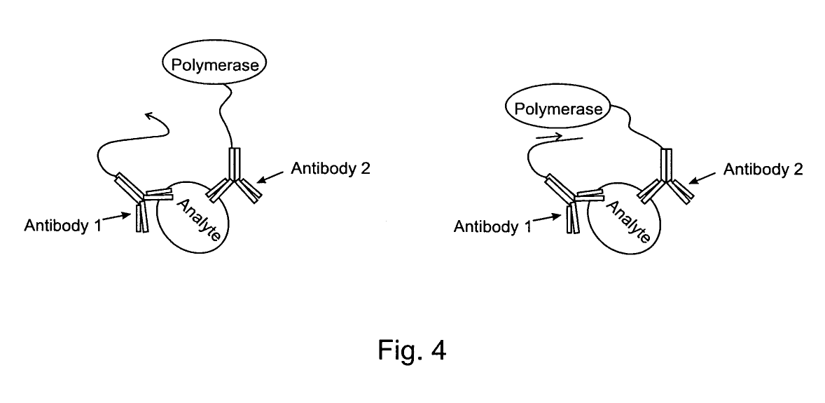

Another aspect of the invention is depicted in FIG. 4. The invention provides a method for detecting an analyte by providing an analyte, a first and second analyte specific probe and a cleavage agent. In one embodiment, the analyte is a polypeptide. In other embodiments, the analyte is an oligonucleotide, cell surface receptor or receptor ligand.

The first analyte specific probe comprises a first binding moiety and an oligonucleotide while the second analyte specific probe comprises a second binding moiety and a polymerase. In one embodiment, the 3′ end of the oligonucleotide is at least partially annealed to a portion of the oligonucleotide and serves as a primer for an extension reaction. In another embodiment, the oligonucleotide is substantially single-stranded and a second oligonucleotide is added which anneals to the first oligonucleotide and serves as a primer for an extension reaction.

In one embodiment, the binding moiety is an antibody. Binding moieties may also include, but are not restricted to, a lectin, cell surface receptor, receptor ligand, peptide, carbohydrate, aptamer, biotin, streptavidin, avidin, protein A and protein G or binding fragments thereof.

The first and the second binding moieties of the probe bind to the analyte. As a result of the two or more probes binding to the analyte, within close proximity to each other, the oligonucleotide and the polymerase interact. The interaction results in the polymerase extending the 3′ end of an oligonucleotide so as to synthesize a nucleic acid strand and form a cleavage site. In one embodiment, the cleavage site is a restriction enzyme cleavage site.

The cleavage agent cleaves the oligonucleotide at the cleavage site thereby releasing a cleavage product. In one embodiment, the cleavage agent is a restriction enzyme. The cleavage product is detected and is indicative of the presence of an analyte. In one embodiment, the release of the cleavage product produces a directly detectable signal, (e.g., a change in a detectable signal, for example upon the cleavage and separation of a FRET pair).

In another embodiment, the released cleavage product is detected by a secondary detection assay. Secondary detection assays are those in which the cleavage product acts as a probe or primer in an assay that produces a detectable signal. Such assays include, but are not limited to, linear and exponential signal detection assays such as those taught in U.S. Pat. Nos. 6,893,819 and 6,348,314, Padlock probe assays such as those disclosed in U.S. Publication No. 2005/0026180, and other methods known in the art and described herein.

In some secondary detection assays the released cleavage product is extended by a polymerase. In other secondary detection assays the released product acts as a probe and may be cleaved by a second cleavage agent.

In a related aspect of the invention, the invention provides kits and compositions for performing the method of single oligonucleotide-polymerase ASP detection method. The kits and compositions include a first analyte specific probe having a first binding moiety and an oligonucleotide. The kits and compositions also include a second analyte specific probe comprising a second binding moiety and a polymerase. The oligonucleotide and the polymerase interact when the first binding moiety and the second binding moiety bind the analyte. The kits and compositions may further comprise one or more additional oligonucleotides.

DNA Binding Domain-Cleavage Agent ASP Detection Method

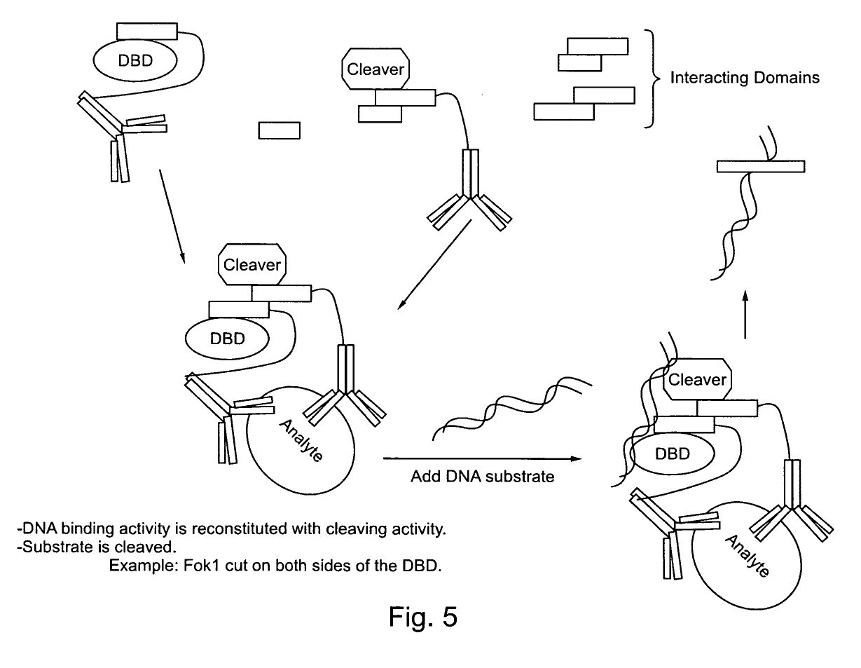

Another aspect of the invention is depicted in FIG. 5. In this aspect a first and a second analyte specific probe, an analyte and a target nucleic acid are incubated in a reaction mixture. In one embodiment, the analyte is a polypeptide. In other embodiments, the analyte is an oligonucleotide, cell surface receptor or receptor ligand.

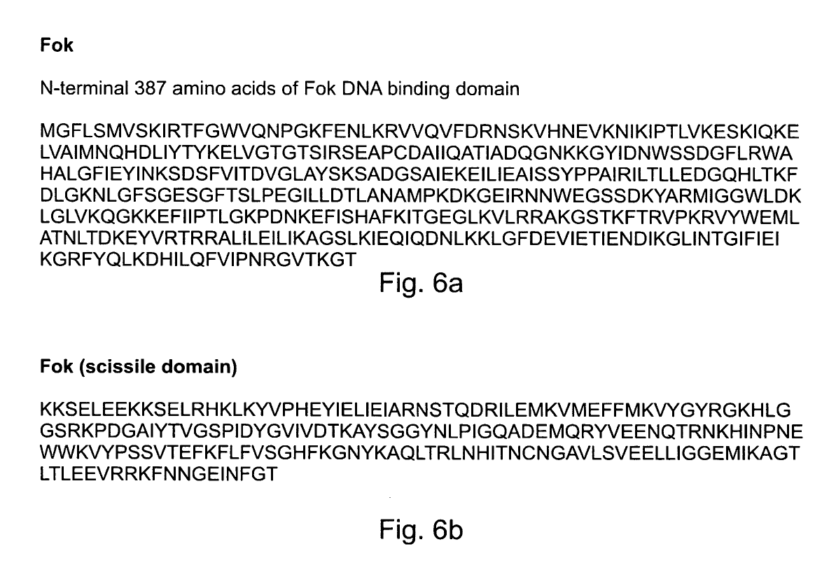

The first analyte specific probe includes a first binding moiety and a DNA binding domain. The DNA binding domain can be any known in the art, and can be derived from a transcription factor, restriction enzyme, or any polypeptide known to interact with a specific DNA sequence. In one embodiment the DNA binding domain is a Ubx homeodomain. In another embodiment, the DNA binding domain is a GAL4 DNA binding domain. In yet another embodiment, the DNA binding domain is AlwI DNA binding domain. In still another embodiment, the DNA binding domain is a Zif-QQR-F_{N} DNA binding domain. In another embodiment, the DNA binding domain is a ZIF-ΔQNK-F_{N} DNA binding domain. In still another embodiment, the DNA binding domain is a FokI DNA Binding Domain (FIG. 6A). The Fok I endonuclease domains and cleavage activity are described in Li and Chandrasegran, Proc. Nat. Acad. Sci. USA; 90(7): 2764-68.

The DNA binding domain is operatively coupled to the binding moiety. The DNA binding domain may be directly or indirectly coupled to the binding moiety. In another embodiment, the DNA binding domain is operatively coupled to a first member of a pair of interacting domains. The DNA binding domain may be directly or indirectly coupled to the first member of the pair of interacting domains.

The second analyte specific probe includes a second binding moiety and a cleavage agent. For example, if the first analyte specific probe includes the Fok I DNA binding domain the second analyte specific probe includes the Fok I cleavage (scissile) domain (FIG. 6B). The cleavage agent is operatively coupled to the binding moiety. The cleaving agent may be directly or indirectly coupled to the binding moiety. In another embodiment, the cleavage agent is operatively coupled to a second member of the pair of interacting domains. The cleavage agent may be directly or indirectly coupled to the second member of the pair of interacting domains.

Pairs of interacting domains can be nucleic acid or polypeptide, or both. Pairs of interacting nucleic acid domains include complementary sequences of nucleic acids between 4 and 30 bases, for example, between 5 and 25 bases, between 6 and 20 bases or between 7 and 15 bases. Pairs of interacting polypeptides can be any known in the art and can include dimerization domains selected from the group consisting of coiled coils, acid patches, zinc fingers, calcium hands, leucine zippers, jun and/or fos (U.S. Pat. No. 5,932,448, incorporated by reference), SH2 (src homology 2), SH3 (src Homology 3; Vidal et al. (2004) Biochemistry. 43, 7336-44, incorporated herein by reference), phosphotyrosine binding (PTB; Zhou et al. (1995) Nature 378:584-592, incorporated herein by reference), WW (Sudol, M. (1996) Prog. Biochys. Mol. Bio. 65:113-132, incorporated herein by reference), PDZ (Kim et al. (1995) Nature 378: 85-88; Komau et al. (1995) Science 269:1737-1740, both incorporated herein by reference) 14.3.3, WD40 (Hu et al., (1998) J Biol Chem. 273, 33489-33494, incorporated herein by reference), and the like. Other interacting domains that can be used include those described, for example, in U.S. patent application Ser. No. 10/613380, incorporated herein by reference in its entirety. The pair of interacting domains can further comprise mutants of these domains in which the binding affinity is altered. Additional pairs of interacting domains can be identified by methods known in the art, including yeast two hybrid screens. Yeast two hybrid screens are described in U.S. Pat. Nos. 5,283,173 and 6,562,576, both of which are herein incorporated by reference in their entireties. Alternatively, a library of peptide sequences can be screened for heterodimerization, for example, using the methods described in WO 01/00814A2, incorporated herein by reference. Useful methods for protein-protein interactions are also described in U.S. Pat. No. 6,790,624, also incorporated herein by reference.

Preferably, the pairs of interacting domains suitable for the present aspect of the invention should not bind to each other at an appreciable level until the first and second binding moieties are bound to the analyte. This is preferable to minimize generation of background by the first and second analyte specific probes that are not bound to the analyte but bind to one another through the interacting domain. Therefore, in one embodiment, the binding affinity of the pair of interacting domains is 10⁻⁸ or higher, for example 3×10⁻⁸, 10⁻⁷, 3×10⁻⁷, 10⁻⁶, 3×10⁻⁶, 10⁻⁵ or higher. Alternatively, the binding of the first and second anal specific probe that are not bound to the analyte (and thus the background generated therefrom) can be reduced by including an excess of one or more of the interacting domains that are not part of an analyte specific probe. Therefore, in another embodiment, one or more of a free domain is added to the reaction mixture. The free domain can be present at an excess, for example 1.1-fold, 1.5-fold, 2-fold, 3-fold, 5-fold, 10-fold, 15-fold, 20-fold, 30-fold, 100-fold excess or more in concentration when compared with the concentration of the first or the second analyte specific probe. The presence of the excess free domain(s) can reduce binding of the first and second analyte specific probe to each other when they are not bound to the analyte. However, when a first and second analyte specific probe are bound to a single analyte, the increased proximity of the first and second probe will favor their interacting, rather than to a free domain.

One can determine whether a pair of interacting domains are suitable for the present aspect of the invention, by performing the DNA binding domain-cleavage agent ASP detection method with and without a known analyte. The method is conducted with the candidate pair of interacting domains in the presence and absence of a known analyte for which a first and second biding moiety are specific. If there is no or little increase in the amount of cleavage products detected in the presence of the analyte, when compared with the reaction mixture without the analyte, the pair of interacting domains is not a good candidate. However, if there is a significant increase, e.g., 2-fold, 3-fold, 4-fold, 5-fold, 10-fold or more increase, in the amount of released cleavage products in the presence of the analyte, when compared with the reaction mixture without the analyte, the pair of interacting domains is a suitable candidate for use in this aspect of the invention.

The binding moieties may be, but are not restricted to, an antibody, a lectin, cell surface receptor, receptor ligand, peptide, carbohydrate, aptamer, biotin, streptavidin, avidin, protein A and protein G or binding fragments thereof.

The first and second binding moieties bind to the analyte. As a result of the probes binding to the analyte, within close proximity to each other, the DNA binding domain and the cleavage agent interact for form a complex. In one embodiment, the cleavage agent is a Fok I nuclease or cleaving fragment thereof. In other embodiments, the cleavage agent is restriction enzyme or nuclease. The cleavage agent cleaves the target nucleic acid at a cleavage site thereby releasing a cleavage product. The cleavage product is a nucleic acid fragment of the oligonucleotide. In one embodiment, the cleavage product is phosphorylated at its 5′ end. In other embodiment, the cleavage products 3′ end capable of being extended by a DNA polymerase.

The cleavage product is indicative of the presence of the analyte. In one embodiment, the release of the cleavage product produces a directly detectable signal, (e.g., a change in a detectable signal, for example upon the cleavage and separation of a FRET pair).

In another embodiment, the released cleavage product is detected by a secondary detection assay. Secondary detection assays are those in which the cleavage product acts as a probe or primer in an assay that produces a detectable signal. Such assays include, but are not limited to, linear and exponential signal detection assays such as those taught in U.S. Pat. No. 6,893,819 and 6,348,314, INVADER technology assays (Third Wave Technologies, Inc, Madison, Wis.), Padlock probe assays such as those disclosed in U.S. Publication No. 2005/0026180, and other methods known in the art and described herein.

In some secondary detection assays the released cleavage product is extended by a polymerase. In other secondary detection assays the released product acts as a probe and may be cleaved by a second cleavage agent.

In a related aspect of the invention, the invention provides kits and compositions for performing the DNA binding domain-cleavage agent ASP detection method. The kits and compositions include a first analyte specific probe comprising a first binding moiety an a DNA binding domain and a second analyte specific probe comprising a second binding moiety and a cleavage agent. The compositions and kits may further include a target nucleic acid.

### Other Embodiments of ASP Detection Methods

Any of the ASP detection methods may be performed in a single reaction tube. In an alternative embodiment, the ASP detection methods are performed in two or more reaction tubes (e.g., ASP binding to analyte and cleavage of oligonucleotide in one reaction tube and detection is a second reaction tube).

In the ASP detection methods the binding moieties and their respective oligonucleotide, cleavage agents, polymerases or activators are operatively coupled to each other. The coupling may be direct or indirect, e.g., requiring linker.

In order to reduce background more than two ASPs may be added to the reaction mixture in any of the methods of the invention. For example, in the single oligonucleotide-cleavage agent ASP detection method two cleavage agents are added to the reaction. In this embodiment, an additional ASP coupled to a second restriction enzyme is added to the assay.

In another embodiment, of the single oligonucleotide-cleavage agent ASP detection method a third ASP is added to the reaction mixture. The third ASP is operatively coupled to an enzyme which phosphorylates a 5′ nucleotide of the cleavage product. The cleavage product can then be used in a subsequent ligation reaction.

In yet another embodiment of the oligonucleotide-cleavage agent ASP detection method, a third ASP is added to the reaction mixture. The third ASP is coupled to an enzyme that unmasks a cleavage site on the first oligonucleotide. In one embodiment, the enzyme is a DNA methylase.

In yet other embodiments of the invention, a first ASP contains a first binding moiety and a first portion of a cleavage agent is added to the reaction mixture. A second ASP contains a second binding moiety and a second portion of a cleavage agent is also added. The first and second portions of the cleavage agents do not have any detectable cleavage activity when they are apart. However, when the first and second ASPs are brought within close proximity to each other, when bound to the analyte, they form a functional cleavage agent. The functional cleavage agent can then cleave a substrate oligonucleotide in the reaction mixture, releasing a cleavage product. In one embodiment, the first and second portions of a cleavage agent are first and second portions of a restriction enzyme that can dimerize to form a functional restriction enzyme upon binding of the first and second binding moieties to the same analyte.

The methods of the invention may also be practiced in a multiplex assay format. In the multiplex assay format several analytes may be simultaneously detected by using several ASP pairs with unique oligonucleotides in order to distinguish them from other pairs. The unique oligonucleotides produce unique cleavage products which can then be detected as indicative of a particular analyte.

Analytes

The invention may be used to detect a wide variety of analytes. It is a requirement, however, that the analytes contain at least two binding moiety binding sites. In this way, at least two analyte specific probes can bind to the same analyte. The binding sites for each ASPs can be the same or different. An analyte can be a single molecule, molecular complex, an organism or virus containing multiple reagent binding sites. Since the length of the oligonucleotides of the ASPs can be constructed to span varying molecular distances, binding sites need not be on the same molecule. However, they may be on separate, but closely positioned, molecules. For example, the multiple binding epitopes of an organism, such as a virus, bacteria or cell can be targeted by the methods of the invention.

Binding Moieties

Binding moieties bind to binding sites within the analyte. The binding moieties can be of the immune or non-immune type. Immune-specific binding-pairs are exemplified by antigen/antibody systems. Antibodies, whether they are polyclonal, a monoclonal or an immunoreactive fragment thereof, can be produced by customary methods familiar to those skilled in the art. Immunoreactive antibody fragment or immunoreactive fragment may be Fab-type fragments which are defined as fragments devoid of the Fc portion, e.g., Fab, Fab′ and F(ab′)2 fragments.

For immune binding moieties, conventional monoclonal and polyclonal antibodies are of use and represent a preferred immune type binding moieties. Established methods of antibody preparation therefore can be employed for preparation of the immune type binding moieties. Suitable methods of antibody preparation and purification for the immune type binding moieties are described in Harlow, Ed and Lane, D in Antibodies a Laboratory Manual, Cold Spring Harbor Laboratory Press, Cold Spring Harbor, N.Y. (1988).

Non-immune binding-moieties include systems, wherein, two components share a natural affinity for each other, but are not antigen/antibody-like pairs. Exemplary non-immune binding-moieties include biotin/avidin or biotin/streptavidin, folic acid-folate binding protein, vitamin B12/intrinsic factor, complementary probe nucleic acids, Proteins A, G, immunoglobulins/, etc. Also included are non-immune binding-pairs that form a covalent bond with each other.

In a one embodiment, different antibodies are used which recognize different epitopes on the analyte. In another embodiment, the antibodies recognize the same epitope on the analyte. Immunoreactive fragments like Fab or F(ab′)2 can also be used. However, the antibodies should be either affinity purified or through other specific adsorbent columns such as protein A.

One could also use non-antibody protein receptors or non-protein receptors such as polynucleic acid aptimers. Polynucleic acid aptimers are typically RNA oligonucleotides which may act to selectively bind proteins, much in the same manner as a receptor or antibody (Conrad et al., Methods Enzymol. (1996), 267(Combinatorial Chemistry), 336-367). Theses aptimers will be suitable in the present invention as binding moieties.

Oligonucleotides of the ASPs

The length of the oligonucleotides of the ASPs can be constructed to span varying molecular distances between analyte binding sites. Thus, the reporter conjugate binding sites need not be on the same molecule but may be located on separate, but closely positioned, molecules within a molecular complex or within an organism. For example, microorganisms, such as viruses and bacteria, could be detected by utilizing the repetitive binding epitopes of the organisms and employing oligonucleotides which span between organism binding epitopes.

The distance between the binding sites need not be precisely known to construct an assay for performing the methods of the invention. Un-hybridized oligonucleotides are flexible. The rotational freedom of the oligonucleotides are further enabled by the flexibility through both the binding moiety and any spacers which may link the oligonucleotides to the binding moieties. Thus, ASPs in different locations and in different configurations are free to interact through molecular motion and can be detected through formation of cleavage products.

To detect binding sites at different molecular distances, the ASP oligonucleotides can be prepared with different lengths. For example, a family of reporter conjugates can be prepared each containing the same binding moiety but different length oligonucleotides. A workable label length for the oligonucleotides can be determined by equilibrating the analyte, in succession, with varying lengths of oligonucleotides, and determining if cleavage products are formed. In this fashion a workable label length for the analyte can be empirically and readily determined. Thus, the distance between the binding sites need not be known to construct an assay for an analyte.

ASP oligonucleotides can be prepared with lengths ranging in length from at least 10 bases in length, typically at least 20 bases in length, for example, at least 30, 40, 50, 60, 70, 80, 90 or 100 bases in length. While the oligonucleotide can be large nucleic acid fragments, it is generally limited to nucleic acids of 500 bases or less.

In conclusion, the flexibility to vary the length of the ASP oligonucleotides can enable the Applicants' invention to be used for detection of a wide range of analytes.

Design and Attachment of ASP Oligonucleotides

The present assay method use oligonucleotide designs whose structures depend on which format is being used to form the cleavage site, overlapping oligonucleotides or a single oligonucleotide with a cleavage enzyme (self-hybridization). In each method the ASP oligonucleotides are each conjugated to a binding moiety. In the assay method using two ASP oligonucleotides each oligonucleotide is designed to be free of duplex formations (dimers, 3′ duplexes or hairpins). In assay methods using one ASP oligonucleotide, (e.g., single oligonucleotide/cleavage enzyme assays), the oligonucleotide is designed to form self-duplexes, 3′ duplexes, hairpins, etc.

Each oligonucleotide has at least a 5′ region and a 3′ region. The size and functional feature of the oligonucleotides depend on the needs of the assay, e.g., distance between binding regions, number of cleavage sites. Each oligonucleotide is designed to form a cleavage site (e.g., restriction enzyme site). In one embodiment, the cleavage site is formed when two oligonucleotides hybridize as a result of ASP binding. In another embodiment, the cleavage site is formed by a single oligonucleotide that self-hybridizes. It is critical that the cleavage site exists when the binding moieties are bound to the analyte.

The oligonucleotides in the two oligonucleotide ASP detection methods consist of two single-stranded oligonucleotides, which are similar in structure. The oligonucleotides have a chemically active group (such as, primary amine group) at any point in its stretch of nucleic acids, which allows it to be conjugated to one of two binding moieties. The two oligonucleotides are sufficiently complementary to form at least a partial overlap when the first and second ASP bind the analyte. The two oligonucleotides must be in close proximity to one another (bound to same analyte) to form the overlap duplex. The minimum length of each oligonucleotide should be long enough to enable the formation this overlapped duplex. Once formed, the duplex will have one or more cleavage sites which are recognized and cleaved by a cleavage enzyme. The cleavage event must release a cleavage product of sufficient size to be detected in a detection step.

The oligonucleotides of the one oligonucleotide formats (e.g., oligonucleotide and cleavage agent) consist of a single nucleic acid which is at least partially self complementary and forms at least a partial duplex when bound to the analyte.

The nucleotide composition of the overlap regions influences the temperature range at which the formation of a stable overlapped duplex occurs. An important criterion for the design of the ASP oligonucleotides is that the nucleotide composition of the overlap region on each ASP will allow for the formation of a stable duplex at temperatures that enables the cleavage agent to cleave the cleavage site. Furthermore, an additional important criterion is that the overlap forms at the temperature the binding moieties are bound to the analyte.

The stringency of the formation of the duplex can be further controlled by adjusting the cation concentration or the concentration of a helix destabilizing agents. Such conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein. For Duplex formation it will be necessary that Hybridization the two oligonucleotides or single self-hybridizing oligonucleotide contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. For hybridizations with shorter ASP oligonucleotides, the position of mismatches becomes more important, and the length of the oligonucleotide determines its specificity (see Sambrook et al., supra, 11.7-11.8).

The ASP oligonucleotides can be attached to the binding moiety at the oligonucleotide's 5′ nucleotide, 3′ nucleotide or at an internal nucleotide. Alternatively, the ASP oligonucleotides are attached indirectly to the binding moiety via streptavidin, protein A or protein G. For example, the ASP oligonucleotides were conjugated to antibodies via biotin-streptavidin. This was performed by conjugation of thiol-modified oligonucleotides to maleimide-derivatized streptavidin, followed by incubation with a biotinylated antibody.

Attachment can be made via direct coupling, or alternatively using a spacer molecule. Linking can be made using any of the means known in the art. Appropriate linking methodologies for attachment oligonucleotides and proteins are described in many references, e.g., Marshall, Histochemical J., 7: 299-303 (1975); Menchen et al., U.S. Pat. No. 5,188,934; Menchen et al., European Patent Application 87310256.0; and Bergot et al., International Application PCT/US90/05565. All are hereby incorporated by reference.

Cleavage Agents

FEN and Nucleases

In one embodiment, the cleavage agent is a nuclease. Nucleases include enzymes that possess 5′ endonucleolytic activity for example a DNA polymerase, e.g. DNA polymerase I from E. coli, and DNA polymerase from Thermus aquaticus (Taq), Thermus thermophilus (Tth), and Thermus flavus (Tfl).

In a further embodiment, the cleavage agent is a FEN nuclease. The term “FEN nuclease” also embodies a 5′ flap-specific nuclease. A cleavage agent according to the invention includes but is not limited to a FEN nuclease enzyme derived from Archaeglobus fulgidus, Methanococcus jannaschii, Pyrococcusfuriosus, human, mouse or Xenopus laevis. A nuclease according to the invention also includes Saccharomyces cerevisiae RAD27, and Schizosaccharomyces pombe RAD2, Pol I DNA polymerase associated 5′ to 3′ exonuclease domain, (e.g. E. coli, Thermus aquaticus (Taq), Thermus flavus (Tfl), Bacillus caldotenax (Bca), Streptococcus pneumoniae) and phage functional homologs of FEN including but not limited to T5 5′ to 3′ exonuclease, T7 gene 6 exonuclease and T3 gene 6 exonuclease. Preferably, only the 5′ to 3′ exonuclease domains of Taq, Tfl and Bca FEN nuclease are used.

A FEN nuclease according to the invention is preferably thermostable. Thermostable FEN nucleases have been isolated and characterized from a variety of thermostable organisms including four archeaebacteria. The cDNA sequence (GenBank Accession No.: AFP03497) and the amino acid sequence (Hosfield et al., 1998a, supra and Hosfield et al., 1998b) for P. furiosus flap endonuclease have been determined. The complete nucleotide sequence (GenBank Accession No.: AB005215) and the amino acid sequence (Matsui et al., supra) for P. horikoshii flap endonuclease have also been determined. The amino acid sequence for M. jannaschii (Hosfield et al., 1998b and Matsui et al., 1999 supra) and A. fulgidus (Hosfield et al., 1998b) flap endonuclease have also been determined.

Restriction Enzymes

In another embodiment, the cleavage agent is a restriction enzyme which selectively cleaves the oligonucleotide of the analyte specific probe. Restriction enzymes bind specifically to and cleave double-stranded DNA at specific sites within or adjacent to a particular recognition sequence. These enzymes have been classified into three groups (e.g. Types I, II, and III) as known to those of skill in the art. The technique of restriction enzyme digestion is well known to those skilled in the art. Reagents useful for restriction enzyme digestion are readily available from commercial vendors including Stratagene, as well as other sources.

DNAzymes and Ribozymes

A DNAzyme is an enzymatic nucleic acid which cleaves both RNA and DNA. DNAzymes can be synthesized chemically or expressed endogenously in vivo, by means of a single stranded DNA vector or an equivalent thereof. DNAzymes are generally reviewed in Usman et al., U.S. Pat. No., 6,159,714; Chartrand et al., 1995, NAR 23, 4092; Breaker et al., 1995, Chem. Bio. 2, 655; Santoro et al., 1997, PNAS 94, 4262; Breaker, 1999, Nature Biotechnology, 17, 422-423; and Santoro et. al., 2000, J. Am. Chem. Soc., 122, 2433-39.

Catalytic nucleic acid can cleave a target nucleic acid substrate provided the substrate meets stringent sequence requirements. The target substrate must be complementary to the hybridizing regions of the catalytic nucleic acid and contain a specific sequence at the site of cleavage. A general model for the DNAzyme has been proposed, and is known as the “10-23” model. DNAzymes following the “10-23” model, also referred to simply as “10-23 DNAzymes”, have a catalytic domain of 15 deoxyribonucleotides, flanked by two substrate-recognition domains of seven to nine deoxyribonucleotides each. In vitro analyses show that this type of DNAzyme can effectively cleave its substrate RNA at purine:pyrimidine junctions under physiological conditions (Santoro and Joyce 1998). Another example of sequence requirements at the cleavage site includes the requirement for the sequence U:X where X can equal A, C or U but not G, for hammerhead ribozymes.

Ribozymes are well characterized in the art and can be optimized for the methods of the present invention. For example, Kramer et al., U.S. Pat. No. 5,616,459, describe a selection method for optimizing a hammerhead or a hairpin ribozyme by mutagenizing the “catalytic domain” of these ribozymes while keeping the binding arm sequence constant. Hammerhead or hairpin ribozymes optimal for cleaving a specific known target site are selected.

Roninson et al., U.S. Pat. No. 5,217,889, and Draper et al., U.S. Pat. No. 5,496,698, describe a method for selecting ribozymes capable of cleaving a known target sequence by fragmenting the DNA of the target gene, inserting the catalytic core of a known ribozyme into these DNA fragments, cloning these fragments into a vector, expressing these ribozymes in a cell and selecting for the vector encoding the optimal ribozyme.

Draper et al., U.S. Pat. No. 5,496,698, also describes a method for identifying ribozyme cleavage sites ′ in a known RNA target by using ribozymes with randomized binding arms.

Additional DNAzyme and ribozyme motifs can be selected for using techniques similar to those described in the references above, and hence, are within the scope of the present invention.

Nickases

Nickases are endonucleases which cleave only a single strand of a DNA duplex. Some nickases introduce single-stranded nicks only at particular sites on a DNA molecule, by binding to and recognizing a particular nucleotide recognition sequence. A number of naturally-occurring nickases have been discovered, of which at present the sequence recognition properties have been determined for at least four. Nickases are described in U.S. Pat. No. 6,867,028, which is herein incorporated by reference in its entirety.

Attachment of Polypeptides to Binding Moieties

Extensive guidance can be found in the literature for covalently linking proteins to binding compounds, such as antibodies, e.g. Hermanson, Bioconjugate Techniques, (Academic Press, New York, 1996), and the like. In one aspect of the invention, one or more proteins are attached directly or indirectly to common reactive groups on a binding moiety. Common reactive groups include amine, thiol, carboxylate, hydroxyl, aldehyde, ketone, and the like, and may be coupled to proteins by commercially available cross linking agents, e.g. Hermanson (cited above); Haugland, Handbook of Fluorescent Probes and Research Products, Ninth Edition (Molecular Probes, Eugene, Oreg., 2002). In one embodiment, an NHS-ester of a molecular tag is reacted with a free amine on the binding compound.

Detection of Cleavage Products

The detection of the cleavage product may be accomplished by several means including (a) direct detection of the released cleavage product on a gel; (b) indirect or direct detection of a change in a signal upon the cleavage of the cleavage site (FRET); (c) hybridization or polymerization of the cleavage product in a subsequent reaction, e.g., sequential amplification, INVADER assays.

The cleavage product is detected and is indicative of the presence of an analyte. In one embodiment, the release of the cleavage product produces a directly detectable signal, (e.g., a change in a detectable signal, for example upon the cleavage and separation of a FRET pair).

In another embodiment, released cleavage product can be detected by a secondary detection assay. Secondary detection assays are assays in which the cleavage product is a probe or primer in a subsequent detection reaction. Such assays include, but are not limited to INVADER technology assays (Third Wave Technologies) and described in U.S. Pat. No. 6,348,314, linear and exponential signal detection assays such as those taught in U.S. Pat. No. 6,893,819, Padlock probe assays such as those disclosed in U.S. Publication No. 2005/0026180, all of which are herein incorporated by reference in their entirety. One of ordinary skill in the art would know of additional suitable assays for detecting the cleavage products of the present invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

### Example 1

The following general discussion of methods, conditions and materials are by way of illustration and not limitation. One of ordinary skill in the art will understand how the methods described herein can be adapted to other applications.

In conducting the methods of the invention, a combination of the assay components is made, including the sample/analyte being tested and two or more ASPs containing a binding moiety and an oligonucleotide and/or a cleavage agent. Generally, assay components may be combined in any order.

The amounts of each reagent are usually determined empirically. The amount of sample/analyte used in an assay will be determined by the predicted number of target analytes present and the means of detection used to monitor the signal of the assay.

In one embodiment, the ASPs are added at a concentration of 5 ug/ml. In specific applications, the concentration used may be higher or lower, depending on the affinity of the binding moieties and the expected number of target molecules present.

The assay mixture is combined and incubated under conditions that provide for binding of the ASPs to the analytes, usually in an aqueous medium, generally at a physiological pH, maintained by a buffer at a concentration in the range of about 10 to 200 mM.

In one embodiment, the incubation temperature for ASP binding is the same temperature for generation of a cleavage product. In another embodiment, the incubation temperature for ASP binding differs from that for generation of a cleavage product. Incubation temperatures normally range from about 4° to 90° C., usually from about 150 to 70° C., more usually 25° to 65°. Typical incubation times can be 15 minutes to 4 hours.

During the incubation step the ASP's are allowed to bind to the analyze and the oligonucleotides are cleaved at cleavage sites by the cleavage agent. The resulting cleavage products are released into solution. The nature of the conditions will depend on the nature of the cleavage agent (e.g., restriction enzyme, FEN, DNAzyme)

Following cleavage, the cleavage products may be detected by any of the methods described herein. Such methods include the INVADER technology assays (Third Wave Technologies). Or any other suitable methods described herein or known in the art. In some instances the detection reaction may be performed in the same reaction mixture. In other embodiments, an aliquot of the cleavage reaction is removed and detected in a separate reaction.

1. A method for detecting an analyte, the method comprising the steps of:
(a) incubating a mixture comprising,
a first analyte specific probe and a second analyte specific probe, wherein said first analyte specific probe comprises a first binding moiety and a DNA binding domain and wherein said second analyte specific probe comprises a second binding moiety and a cleavage agent,
a target nucleic acid having a cleavage site, and
an analyte,
so as to permit said first and said second binding moieties to bind to said analyte such that said DNA binding domain and said cleavage agent interact to form a complex comprising said DNA binding domain and said cleavage agent and so as to permit cleavage of said target nucleic acid at said cleavage site with said cleavage agent, thereby releasing a cleavage product; and
(b) detecting said cleavage product, wherein said detection of said cleavage product is indicative of the presence of said analyte.

2. The method of claim 1, wherein said DNA binding domain is operatively coupled to a first member of a pair of interacting domains and said cleavage agent is operatively coupled to a second member of said pair of interacting domains, wherein said complex is formed when said first and second interacting domains bind to each other.

3. The method of claim 1, wherein said cleavage agent is monomer restriction enzyme or nuclease.

4. The method of claim 1, wherein said cleavage agent is a Fok I nuclease or fragment thereof.

5. The method of claim 1, wherein said binding moiety is selected from the group consisting of a monoclonal antibody, polyclonal antibody, lectin, cell surface receptor, receptor ligand, peptide, carbohydrate, aptamer, biotin, streptavidin, avidin, protein A and protein G or binding fragments thereof.

6. The method of claim 1, wherein said analyte is selected from the group consisting of a protein, oligonucleotide, cell surface receptor and receptor ligand.

7. A method for detecting an analyte, the method comprising the steps of:
(a) incubating a mixture comprising,
a first analyte specific probe and a second analyte specific probe, wherein said first analyte specific probe comprises a first binding moiety and a first portion of a cleavage agent and wherein said second analyte specific probe comprises a second binding moiety and a second portion of a cleavage agent,
a target nucleic acid having a cleavage site, and
an analyte,
so as to permit said first and said second binding moieties to bind to said analyte such that said first portion of said cleavage agent and said second portion of said cleavage agent interact to form a functional cleavage agent so as to permit cleavage of said target nucleic acid at said cleavage site with said cleavage agent, thereby releasing a cleavage product; and
(b) detecting said cleavage product, wherein said detection of said cleavage product is indicative of the presence of said analyte.

8. The method of claim 7, wherein said cleavage agent is selected from the group consisting of a restriction enzyme, a nuclease, and a FEN nuclease.

9. The method of claim 7, wherein said cleavage site is selected from the group consisting of a restriction enzyme cleavage site, a nuclease cleavage site, and a FEN nuclease cleavage site.

10. The method of claim 7, wherein said cleavage product is phosphorylated at its 5′ end.

11. The method of claim 7, wherein said release of said cleavage product produces a detectable signal.

12. The method of claim 7, wherein said released cleavage product is detected in a sequential amplification reaction.

13. The method of claim 7, wherein said released cleavage product anneals to an oligonucleotide.

14. The method of claim 13, wherein said cleavage product acts as a primer in a subsequent cleavage reaction.

15-33. (canceled)

34. A composition for detecting an analyte, said composition comprising:
a first analyte specific probe comprising a first binding moiety and an oligonucleotide
a second analyte specific probe comprises a second binding moiety and a second oligonucleotide
wherein said first and second binding moieties bind to said analyte to form a cleavage site.

35. A composition for detecting an analyte, said composition comprising:
a first analyte specific probe comprising a first binding moiety and an oligonucleotide having a cleavage site; and
a second analyte specific probe comprises a second binding moiety and a cleaving agent,
wherein said first and second binding moieties bind to said analyte to form a complex comprising the oligonucleotide and said cleaving agent.

36. A composition for detecting an analyte, said composition comprising:
a first analyte specific probe comprising a first binding moiety and an oligonucleotide having a cleavage site, cleavage activity and activator binding site; and
a second analyte specific probe comprises a second binding moiety and an activator,
wherein said first and second binding moieties bind to said analyte to allow said activator and said oligonucleotide to interact, wherein said interaction allows the activator to bind the activator binding site and activate said cleavage activity.

37. A composition for detecting an analyte, said composition comprising:
a first analyte specific probe comprising a first binding moiety and an oligonucleotide; and
a second analyte specific probe comprises a second binding moiety and a polymerase,
wherein said first and second binding moieties bind to said analyte such that said oligonucleotide and said polymerase interact.

38. A kit for detecting an analyte, said kit comprising:
a first analyte specific probe comprising first binding moiety and a DNA binding domain;
a second analyte specific probe comprising a second binding moiety and a cleavage agent; and
wherein said first and said second binding moieties bind to said analyte such that said DNA binding domain and said cleavage agent interact to form a complex comprising said DNA binding domain and said cleavage, and packaging material therefore.

39. A composition for detecting an analyte, said composition comprising:
a first analyte specific probe comprising first binding moiety and a DNA binding domain;
a second analyte specific probe comprising a second binding moiety and a cleavage agent; and
wherein said first and said second binding moieties bind to said analyte such that said DNA binding domain and said cleavage agent interact to form a complex comprising said DNA binding domain and said cleavage.

40. A kit for detecting an analyte, said kit comprising:
a first analyte specific probe comprising a first binding moiety and a first portion of a cleavage agent;
a second analyte specific probe comprising a second binding moiety and a second portion of a cleavage agent; and
wherein said first and said second binding moieties bind to said analyte so as to permit said first and said second binding moieties to bind to said analyte such that said first portion of said cleavage agent and said second portion of said cleavage agent interact to form a functional cleavage agent, and packaging material therefore.

41. A composition for detecting an analyte, said composition comprising:
a first analyte specific probe comprising a first binding moiety and a first portion of a cleavage agent;
a second analyte specific probe comprising a second binding moiety and a second portion of a cleavage agent; and
wherein said first and said second binding moieties bind to said analyte so as to permit said first and said second binding moieties to bind to said analyte such that said first portion of said cleavage agent and said second portion of said cleavage agent interact to form a functional cleavage agent.
